(19) United States
(12) Patent Application Publication
(10) Pub. No.: US 2010/0041607 A1
(43) Pub. Date: Feb. 18, 2010

(54) UNCTUOUS COMPOSITIONS

(75) Inventors: Jesper Feldthusen Jensen, Nieder-Olm (DE); Christian Köpsel, Weinheim (DE); Helmut Auweter, Limburgerhof (DE); Ingrid Martin, Ludwigshafen (DE); Angelika-Maria Pfeiffer, Lambrecht (DE); Erik Lüddecke, Mutterstadt (DE); Dieter Feuerstein, Dudenhofen (DE)

Correspondence Address: CONNOLLY BOVE LODGE & HUTZ, LLP, P O BOX 2207, WILMINGTON, DE, 19899, US

(73) Assignee: BASF SE, Ludwigshafen (DE)

(21) Appl. No.: 12/522,558

(22) PCT Filed: Jan. 15, 2008
(86) PCT No.: PCT/EP08/50399

§ 371 Date: Jul. 9, 2009

(30) Foreign Application Priority Data
Jan. 16, 2007 (EP) 07100632.4
Mar. 30, 2007 (EP) 07105359.9

(51) Int. Cl. A61K 38/16; A61K 31/122; A61P 43/00; A61Q 90/00

(52) U.S. Cl. 514/12; 514/691

(57) ABSTRACT

The present invention relates to unctuous composition containing at least one solid active ingredient, at least one hydrophobic protective colloid and at least one edible oil. The active ingredient is preferably a carotenoid. The preferred protective colloids are prolamines. The invention compositions are simple to produce, have good bioavailability and dyestuff yield, and are used an addition to animal feed agents, foods, and dietary supplements and pharmaceutical and cosmetic agents.

The present invention relates to oily formulations, method for their production, and their use as addition to animal feeds and human foods.

The formulation of carotenoids represents a particular challenge because of their slight solubility in water and their chemical instability. There have thus been numerous attempts to provide carotenoid formulations which, on the one hand, are stable and, on the other hand, show good bioavailability and provide the desired color yield on use. Liquid carotenoid formulations are of particular interest because the obtaining of the carotenoids in powder form and the preparation of a liquid formulation by the user become superfluous.

Production of carotenoid primary particles with a particle size in the nanometer range is crucial for achieving adequate bioavailability and color yields. Two methods in particular have been disclosed for producing such particles:

(1) An emulsification/precipitation process (micronization) as described for example in EP 410 236 A. According to EP 065 193 A, finely divided carotenoid products in powder form are produced by dissolving a carotenoid in a volatile, water-miscible organic solvent at elevated temperature and, if appropriate, under elevated pressure, precipitating the carotenoid by mixing with an aqueous solution of a protective colloid, concentrating and subsequently spray drying. EP 1 213 013 A describes the production of an aqueous carotenoid dispersion in the presence of a hydrocolloid such as casein or gelatin. In this case, the active ingredient is flocculated together with the hydrocolloid by adjusting the pH to the isoelectric point, and subsequently the flocculated material is separated off and dried. Dispersing the resulting product in powder form then affords an oily suspension. An analogous method using water-immiscible solvents is described in EP 937 412 A.

(2) A size-reduction process, in particular by grinding, as described for example in WO 91/06292 and WO 94/19411. The grinding of the carotenoids takes place using a colloid mill, in aqueous or oily media, attaining a particle size in the nanometer range.

WO 96/23429 describes the production of oily astaxanthin suspensions with particle sizes of <2 μm by grinding astaxanthin, adding an oil during or after the grinding. The use of protective colloids or emulsifiers is not disclosed. It is pointed out that the particles are prone to agglomeration. Accordingly, further stabilization by storing the suspension below the solidification temperature is considered.

WO 031102116 describes oily solutions of a carotenoid. These oily solutions are produced by dissolving the carotenoids in an organic solvent such as N-methyl-pyrrolidone in the presence of a lipophilic dispersant and removing the solvent. The resulting powder is then dissolved in low concentration in an oil, e.g. fish oil.

WO 2006/125591 describes the production of an oily carotenoid composition. In this case, the carotenoid is dispersed in an oil phase, the dispersion is heated for a short time in order to dissolve the carotenoid, and the resulting solution is cooled by mixing with further oil at a lower temperature than the oil solution. The oily composition comprises only a small amount of carotenoid, i.e. large amounts of the composition must be employed in order to achieve the desired effect. The method is therefore uneconomic.

WO 96/13178 describes the production of stable lycopene concentrates by grinding lycopene in a liquid medium in which the lycopene is essentially insoluble. The liquid medium used is glycerol, propylene glycol or ethanol.

It is common to prior art methods that either they result in products with unsatisfactory stability or bioavailability, or that the production is complicated.

The present invention was therefore based on the object of providing liquid carotenoid formulations which can easily be produced and nevertheless result in products whose stability and bioavailability are comparable to known products.

It has now been found, surprisingly, that this object is achieved by an oily formulation which comprises a solid active ingredient and a hydrophobic protective colloid dispersed or dissolved in an edible oil.

The present invention therefore relates to an oily formulation in the form of a dispersion (suspension) comprising at least one active ingredient, at least one hydrophobic protective colloid and at least one edible oil.

Solid active ingredients which can be dispersed in the edible oil are suitable as active ingredient. The active ingredients generally have a particle size in the range from 50 nm to 10 μm, preferably 100 nm to 5 μm, particularly preferably 100 nm to 3 μm, 150 nm to 2 μm and in particular 200 nm to 1 μm.

The active ingredients are preferably carotenoids. It is possible in this connection to employ the known representatives which are obtainable from natural sources or by synthesis. Examples thereof are β-carotene, lycopene, lutein, astaxanthin, astaxanthin derivatives (such as, for example, astaxanthin dimethyl disuccinate or astaxanthin dipalmitate), zeaxanthin, cryptoxanthin, citranaxanthin, canthaxanthin, echinenone, bixin, β-apo-4-carotenal, β-apo-8-carotenal, β-apo-4-carotenoic esters, singly or as mixture. Astaxanthin, astaxanthin derivatives (such as, for example, astaxanthin dimethyl disuccinate or astaxanthin dipalmitate), β-carotene, β-apo-8-carotenal, ethyl β-apo-8′-carotenoate, canthaxanthin, citranaxanthin, echinenone, lutein, lycopene and zeaxanthin are preferred. Astaxanthin, astaxanthin derivatives (such as, for example, astaxanthin dimethyl disuccinate or astaxanthin dipalmitate) and canthaxanthin are particularly preferred, especially astaxanthin.

The formulations of the invention additionally comprise a hydrophobic protective colloid. A hydrophobic protective colloid means a protective colloid which is insoluble in water, cannot be dispersed in water and is not swellable. It is, however, soluble in aqueous ethanol or isopropanol comprising at least 40% by weight ethanol or isopropanol. The protective colloid is regarded as soluble if more than 0.5% by weight of the hydrocolloid dissolves in aqueous ethanol or isopropanol with at least 40% by weight alcohol. The protective colloids have affinity for phenyl-, octyl- or butyl-Sepharose.

A preferred group of hydrophobic protective colloids are the prolamines. These are proteins which occur in cereals species. Examples of suitable prolamines are zein (from maize), gliadin (from wheat and oats), secalin (from rye), hordein (from barley), orycin (from rice) and kafarin (from millet, sorghum).

Also suitable are hydrophobic protective colloids, especially proteins, which have been produced and/or modified by fermentation, or which have been prepared by synthesis.

The edible oil may be of synthetic, mineral, vegetable or animal origin. Examples are sesame oil, corn oil, cottonseed oil, soybean oil, peanut oil, esters of medium-chain vegetable fatty acids, oleostearin, liquid paraffin, glyceryl stearate, isopropyl myristate, diisopropyl adipate, cetylstearyl 2-ethylhexanoate, hydrogenated polyisobutene, caprylic acid/cabric acid triglycerides, palm oil, palm kernel oil, lanolin and PUFAs (polyunsaturated fatty acids, such as eicosapentaenoic acid (EPA), docosahexaenoic acid (DHA) and alpha-linolenic acid.

Preferred edible oils are liquid at 30° C., such as sunflower oil, palm oil, palm kernel oil, sesame oil, corn oil, cottonseed oil, soybean oil, peanut oil, esters of medium-chain triglycerides (MCT oils as they are known), fish oils such as mackerel, sprat or salmon oil. Particularly preferred for livestock nutrition are fish oils, corn oil, sunflower oil, soybean oil and peanut oil. The oils advantageous for the food/drugs sector are those mentioned for livestock nutrition, plus the esters of medium-chain triglycerides.

The amount of the active ingredients present in the formulations of the invention is generally in the range from 0.1 to 50% by weight, preferably 0.2 to 30% by weight and in particular 1.0 to 15% by weight, based on the total weight of the oily formulation.

The amount of hydrophobic protective colloid is generally in the range from 1 to 75% by weight, preferably 2 to 70% by weight and in particular 5 to 65% by weight, based on the total weight of the formulation.

The ratio of active ingredient to hydrophobic protective colloid is generally in the range from 5:1 to 1:10, preferably 3:1 to 1:5, by weight.

The amount of edible oil is generally in the range from 20 to 98.9% by weight, preferably 30 to 98% by weight, particularly preferably 40 to 97% by weight, and in particular 50 to 95% by weight, based on the total weight of the oily formulation.

To increase the stability of the active ingredient in relation to oxidative degradation, it is advantageous to add stabilizers such as α-tocopherol, t-butylhydroxytoluene, t-butylhydroxyanisole, ascorbic acid or ethoxyquin.

The formulations of the invention may additionally comprise ancillary substances such as thickeners, hard fats, chelating agents, for example alkali metal or alkaline earth metal salts of citric acid, phythic acid or phosphoric acid and/or emulsifiers. Examples of emulsifiers are ascorbyl palmitate, polyglycerol fatty acid esters such as polyglycerol 3 polyricinoleate (PGPR 90), sorbitan fatty acid esters such as sorbitan monostearate (span 60), PEG (20) sorbitol monooleate, propylene glycol fatty acid esters or phospholipids such as lecithin.

The formulations of the invention can be produced by a plurality of methods. In a first method, the active ingredient is ground with the oil used. The hydrophobic protective colloid can be present wholly or partly from the outset or be added during the grinding. It can be added in powdered form or as solution. If it is added as solution, the solvent expediently used is a mono- or polyalcohol, if appropriate mixed with water, or a carboxylic ester or mixtures thereof. Examples of suitable solvents are isopropanol/water (8:2), propylene glycol, ethylene glycol, triethylene glycol or ethyl lactate. The grinding is continued until an average particle size of the active ingredient crystals and of the hydrophobic protective colloid of <5 μm, preferably <1 μm, is reached. It is possible to employ as grinding apparatus the usual apparatuses known to the skilled worker, for example colloid mills, ball mills such as Dynomill of the KDL type etc.

It may be expedient to add a small amount of water or alkanol or a mixture thereof to the mixture to be ground. Suitable alkanols are for example methanol, ethanol, isopropanol, etc. In general, not more than 10% by weight of water, alkanol or a mixture thereof are added to the mixture to be ground.

In a further method, the solid active ingredient and the hydrophobic protective colloid are dissolved in a polar solvent. The polar solvent used is in particular water-miscible, thermally stable, volatile solvents comprising only carbon, hydrogen and oxygen, such as alcohols, ethers, esters, ketones and acetates. The solvents expediently used are those which are at least 10% miscible with water, have a boiling point below 200° C. and/or have fewer than 10 carbon atoms. Methanol, ethanol, n-propanol, isopropanol, 1,2-butanediol 1-methyl ether, 1,2-propanediol n-propyl ether, tetrahydrofuran or acetone is particularly preferably used. A mixture of these solvents with water can also be used, in which case the amount of solvent is at least 40% by weight. Examples of such mixtures are mixtures of ethanol or isopropanol with water.

The active ingredient and the hydrophobic protective colloid are dissolved in the solvent used by initially dispersing the two components in the solvent at a temperature in the range from room temperature up to approximately the boiling point of the solvent used. The dispersion is subsequently mixed with further solvent, which is heated to a higher temperature, thus dissolving the components. The temperature of the further solvent is generally in the range from 150 to 250° C., operating under a pressure which is set up at the temperature used. Immediately thereafter (within 0.1 to 30 seconds), the resulting solution is mixed with water, resulting in a dispersion of the active ingredient particles in the solvent-water mixture. The particle size of the active ingredient particles is generally in the range from 50 nm to 800 nm. The amount of added water is generally from 1 to 10 times the amount of the solvent used. The pH of the added water is adjusted so that it is at least 2 pH units different from the isoelectric point of the protective colloid used. For example, on use of zein, the pH is adjusted to a value of at least 9 and in particular to a value of at least 10.

There are two alternatives for further processing. The solvent is removed from the resulting active ingredient dispersion in a conventional way, or the active ingredient/-protective colloid particles are induced to flocculate. This takes place by adjusting the pH of the active ingredient dispersion to the isoelectric point of the protective colloid. The flocculated particles, which represent an active ingredient-hydrophobic protective colloid aggregate, are then isolated in a conventional way, for example by filtration, centrifugation or spray drying, and dried if appropriate. A liquid formulation is obtained by taking up the resulting product in powder form in the desired edible oil.

Details of the method and further variants of the method are described in EP 1 213 013 and EP 1 219 292, the disclosure of which is included in its entirety in the present invention.

The formulations of the invention can be diluted before use to the respective use concentration by adding fats or oils. The dilution can take place for example by stirring at elevated temperature such as 30 to 80° C.

The formulations are suitable inter alia as additive to animal feeds and human food preparations or compound feed, as compositions for producing pharmaceutical and cosmetic preparations, and for producing dietary supplement products in the human and animal sector. The suspensions can preferably be employed as addition to feed in livestock nutrition, for example by mixing into feed pellets on extrusion or by application or spraying onto feed pellets. Use as addition to feed takes place in particular by direct spraying on of the formulations of the invention, for example as so-called post-pelleting application. The feed pellets are preferably loaded with the formulations under reduced pressure.

Typical areas of use in the human food sector are for example the vitaminization and coloring of beverages, dairy products such as yoghurt, milk drinks or milk ice, and blancmange powders, egg products, baking mixes and confectionery. In the cosmetics sector, the oily suspensions can be used for example for decorative personal care compositions, for example in the form of a cream, of a lotion, as lipstick or makeup.

The invention further relates to dietary supplements, animal feeds, human foods and pharmaceutical and cosmetic preparations which comprise the formulations of the invention. Preference is given to animal feeds, especially feed pellets, loaded with the formulations.

Dietary supplement products and pharmaceutical preparations mean inter alia tablets, coated tablets and, preferably, hard and soft gelatin capsules which comprise the formulations of the invention.

The following examples illustrate the invention without limiting it.

### EXAMPLE 1

### Production of an Oily Astaxanthin Dispersion

Production took place in an apparatus of the type described in EP 065 193. 40 g of crystalline astaxanthin, 60 g of zein, 2 g of ascorbyl palmitate and 4 g of ethoxyquin were suspended in 600 g of an isopropanol/water mixture (60:40) at room temperature in a heatable receiver at a temperature of 30° C. The active ingredient suspension was then heated to 87.8° C. and mixed continuously, at a flow rate of 2.14 kg/h, with further isopropanol/water mixture (60:40) which was at a temperature of 290° C. with a flow rate of 3.0 kg/h. A mixing temperature of 170° C. and a pressure of 54.8 bar were set up. The resulting solution was immediately thereafter (<1 sec) mixed with 16 643 g of distilled water whose pH had been adjusted to pH 11 with 1 M NaOH, at a flow rate of 50.5 kg/h, resulting in a colloidal active ingredient dispersion.

The active ingredient particles exhibit a particle size of 150 nm in the isopropanol/water mixture at an E 1/1 of 110.

The pH of the dispersion was then adjusted to pH 5.1 with 1 M HCl in order to flocculate the astaxanthin/zein particles. The flocculated particles were dried and dispersed in soybean oil in such a way that a 9.5% by weight astaxanthin dispersion was obtained. The active ingredient content of this dispersion remained unchanged over a period of 6 months. The oily dispersion can be applied directly by dilution with oil to feed pellets.

### EXAMPLE 2 (COMPARATIVE)

### Production of an Astaxanthin Formulation Using a Hydrophilic Protective Colloid

83.5 g of crystalline astaxanthin and 20 g of α-tocopherol were suspended in 626 g of an azeotropic isopropanol/water mixture at room temperature in a heatable receiver at a temperature of 30° C. in analogy to the method described in example 1. The active ingredient suspension was then heated to 90° C. and mixed continuously, at a flow rate of 2.1 kg/h, with further isopropyl/water azeotrope which was at a temperature of 220° C., and a flow rate of 2.6 kg/h, the astaxanthin dissolving at a mixing temperature of 165° C. and a pressure of 55 bar. This active ingredient solution was immediately thereafter mixed with an aqueous phase of a solution of 83.5 g of soybean protein and 177 g of lactose in 11 010 g of distilled water, in which the pH had been adjusted to pH 9.5 with 1 M NaOH, at a flow rate of 32.5 kg/h.

The active ingredient particles resulting in the mixing exhibited a particle size of 107 nm in the isopropanol/water mixture at an E 1/1 of 124.

The dispersion was then concentrated in a thin-film evaporator to a dry content of about 23.7% by weight and finally spray dried. The dry powder had an astaxanthin content of 23% by weight. The dried powder redispersed in water had a particle size of 317 nm and exhibited an E 1/1 of 101. The E 1/1 is the specific extinction of a 0.5% by weight dispersion of a 20% by weight dry powder in a 1 cm cuvette at the absorption maximum.

The resulting astaxanthin powder could not be dispersed homogeneously in an oil. In order to be able to apply this powder to feed pellets it is necessary first for the powder to be dissolved in water and then for the aqueous solution to be emulsified into an oil.

### EXAMPLE 3

### Production of an Oily Astaxanthin Formulation by Grinding

50 g of astaxanthin (5% by weight), 40 g of α-tocopherol (4% by weight) and 5 g of ascorbyl palmitate (0.5% by weight) are mixed with 755 g of sunflower oil (75.5% by weight). This mixture was ground and, during the first 15 minutes, 150 g of zein (15% by weight) were slowly added. Grinding time: 4 hours at 64 to 74° C. and a flow rate of 60 to 80 g/min (specifications of the Dynomill mill: 0.05 mm grinding slit, 2986 rpm, 470 ml with ZrO₂ stabilized beads with a diameter of 0.3 mm). A particle size of D 4,3=9.5 μm was measured by Fraunhofer diffraction for the astaxanthin-zein agglomerate. The astaxanthin primary particles were considerably smaller, and the size of the astaxanthin crystals determined by transmission electron microscopy was 200 to 600 nm.

1.-11. (canceled)

12. An oily formulation comprising at least one solid active ingredient, at least one hydrophobic protective colloid and at least one edible oil.

13. The formulation according to claim 12, wherein the active ingredient is a carotenoid.

14. The formulation according to claim 12, wherein the active ingredient is astaxanthin.

15. The formulation according to claim 12, wherein the hydrophobic protective colloid is a prolamine.

16. The formulation according to claim 15, wherein the prolamine is zein, gliadin, zein, kafarin, secalin, hordein or orycin.

17. The formulation according to claim 12, wherein the ratio of active ingredient to hydrophobic protective colloid is in the range from 5:1 to 1:10 by weight.

18. The formulation according to claim 14, wherein the hydrophobic protective colloid is a prolamine.

19. The formulation according to claim 18, wherein the prolamine is zein, gliadin, zein, kafarin, secalin, hordein or orycin.

20. The formulation according to claim 19, wherein the ratio of active ingredient to hydrophobic protective colloid is in the range from 5:1 to 1:10 by weight.

21. A method for producing the formulation according to claim 12, which comprises grounding the solid active ingredient in at least one edible oil in the presence of at least one hydrophobic protective colloid.

22. A method for producing the formulation according to claim 12, which comprises
a) dispersing the solid active ingredient and the hydrophobic protective colloid in a polar solvent,
b1) removing the solvent or
b2) flocculating the active ingredient together with the hydrophobic protective colloid and the flocculated solid is obtained, and
c) dispersing the solid obtained as in b1) or b2) in an oil.

23. The method according to claim 23, wherein the solvent is a mixture of water and a water-miscible alcohol.

24. An animal feed, human food or dietary supplement comprising the oily formulation according to claim 12.

25. A pharmaceutical agent comprising the oily formulation according to claim 12.

26. A cosmetic agent comprising the oily formulation according to claim 12.
